(19) United States
(12) Patent Application Publication
(10) Pub. No.: US 2022/0401933 A1
(43) Pub. Date: Dec. 22, 2022

(54) ZEOLITE CATALYZED PROCESS FOR THE AMINATION OF PROPYLENE OXIDE

(71) Applicant: BASF SE, Ludwigshafen am Rhein (DE)

(72) Inventors: Alexander Michael HAYDL, Ludwigshafen am Rhein (DE); Alvaro GORDILLO BOLONIO, Ludwigshafen am Rhein (DE); Andrei-Nicolae PARVULESCU, Ludwigshafen am Rhein (DE); Johann-Peter MELDER, Ludwigshafen am Rhein (DE); Ulrich MUELLER, Neustadt (DE); Thomas KRUG, Ludwigshafen am Rhein (DE)

(21) Appl. No.: 17/777,759

(22) PCT Filed: Nov. 19, 2020
(86) PCT No.: PCT/EP2020/082665

§ 371 Date: May 18, 2022

(30) Foreign Application Priority Data
Nov. 20, 2019 (EP) 19210400.8

(51) Int. Cl. B01J 29/40; B01J 29/46; C07C 213/04

(52) U.S. Cl. CPC B01J 29/405; B01J 29/46; C07C 213/04

(57) ABSTRACT

The present invention relates to a process for the conversion of propylene oxide to 1-amino-2-propanol and/or di(2-hydroxypropyl)amine comprising (i) providing a catalyst comprising a zeolitic material comprising YO₂ and optionally comprising X₂O₃ in its framework structure, wherein Y is a tetravalent element and X is a trivalent element, wherein the zeolitic material has a framework-type structure selected from the group consisting of MFI and/or MEL, including MEL/MFI intergrowths; (ii) providing a mixture in the liquid phase comprising propylene oxide and ammonia; (iii) contacting the catalyst provided in (i) with the mixture in the liquid phase provided in (ii) for converting propylene oxide to 1-amino-2-propanol and/or di(2-hydroxypropyl)amine.

### TECHNICAL FIELD

The present invention relates to a process for the conversion of propylene oxide to 1-amino-2-propanol and/or di(2-hydroxypropyl)amine using a zeolite catalyst having a framework-type structure selected from the group consisting of MFI and/or MEL, including MEL/MFI intergrowths.

### INTRODUCTION

In principle, isopropanolamines are produced as a product mixture between monoiso- (MIPOA), diiso- (DIPOA) and triisopropanolamine (TIPOA). Depending on the molar ratio of ammonia (NH₃) to propylene oxide (PO) the product distribution can be adjusted to a certain extent.

U.S. Pat. No. 3,697,598 A describes the production of monoalkanolamines, by reacting an alkylene oxide, and in particular propylene or ethylene oxide, using a cation exchange resin catalyst. U.S. Pat. No. 5,599,999 A relates to the production of monoalkanolamines, and in particular of ethanolamine, using an inorganic solid catalyst comprising a rare earth element supported on an inorganic heat-resistant carrier. U.S. Pat. No. 4,438,281 A concerns the production of monoalkanolamines, and in particular of ethanolamine, using an inorganic catalyst, and in particular silica-alumina. EP 0375267 A2 describes to the production of alkanolamines, and in particular of ethanolamine, using an acidic montmorillonite clay catalyst. CN 101884934 A relates to a ZSM-5 zeolite-based process for the production of ethanolamine.

Despite the progress achieved relative to the amination of alkylene oxides, there remains the need for a process and a catalyst which displays both an improved activity and selectivity in the amination reactions, in particular towards the mono- and dialkylated amine products, and yet more towards the monoalkylated amine products. In particular, there remains a need for a process and a catalyst, wherein the conversion of the alkylene oxide educts is practically complete, and wherein the production of the unwanted trialkylated amine products may be reduced to an absolute minimum, if not practically eliminated from the product spectrum.

### DETAILED DESCRIPTION

It was therefore an object of the present invention to provide a process for the amination of alkylene oxides, and in particular of propylene oxide with ammonia, with an improved efficiency relative to the conversion of propylene oxide, and which furthermore displays a high selectivity towards monoisopropanol amine, and a low selectivity towards triisopropanolamine amine. Said object is achieved by the inventive process. Thus, it has surprisingly been found that by specifically using a zeolitic catalyst material having a framework-type structure selected from the group consisting of MFI and/or MEL, including MEL/MFI intergrowths, a highly improved process for the amination of propylene oxide may be obtained displaying superior results both with regard to the activity as well as with regard to the selectivity of the amination reaction. In particular it has quite unexpectedly been found that in the amination of propylene oxide, the selectivity of the reaction toward monoisopropanol may be substantially increased, wherein at the same time practically no triisopropanolamine side product is produced when employing the inventive process.

Therefore, the present invention relates to a process for the conversion of propylene oxide to 1-amino-2-propanol and/or di(2-hydroxypropyl)amine comprising

(i) providing a catalyst comprising a zeolitic material comprising YO₂ and optionally comprising X₂O₃ in its framework structure, wherein Y is a tetravalent element and X is a trivalent element, wherein the zeolitic material has a framework-type structure selected from the group consisting of MFI and/or MEL, including MEL/MFI intergrowths;
(ii) providing a mixture in the liquid phase comprising propylene oxide and ammonia;
(iii) contacting the catalyst provided in (i) with the mixture in the liquid phase provided in (ii) for converting propylene oxide to 1-amino-2-propanol and/or di(2-hydroxypropyl)amine.

It is preferred that the zeolitic material has an MFI or an MEL/MFI intergrowth framework-type structure. It is particularly preferred that the zeolitic material has an MFI framework-type structure.

It is preferred that the catalyst provided in (i) comprises a zeolitic material having an MFI framework-type structure, wherein the zeolitic material preferably comprises one or more zeolites selected from the group consisting of Silicalite, ZSM-5, [Fe—Si—O]-MFI, Monoclinic H-ZSM-5, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, organic-free ZSM-5, and mixtures of two or more thereof, more preferably from the group consisting of ZSM5, AMS-1B, AZ-1, FZ-1, LZ-105, NU-4, NU-5, TS-1, TSZ, TSZ-III, TZ-01, USC-4, USI-108, ZBH, ZKQ-1B, ZMQ-TB, and mixtures of two or more thereof, wherein more preferably the zeolitic material comprises TS-1 and/or ZSM-5, wherein more preferably the zeolitic material is TS-1 and/or ZSM-5.

As disclosed above, it is preferred that the catalyst provided in (i) comprises a zeolitic material having an MEL/MFI intergrowth framework-type structure. In the case where the catalyst provided in (i) comprises a zeolitic material having an MEL/MFI intergrowth framework-type structure, it is preferred that the zeolitic material comprises Bor-D and/or ZBM-10, preferably ZBM-10, wherein more preferably the zeolitic material is ZBM-10.

In accordance with the above, it is preferred that the catalyst provided in (i) comprises a zeolitic material having an MEL framework-type structure. In the case where the catalyst provided in (i) comprises a zeolitic material having an MEL framework-type structure, it is preferred that the zeolitic material comprises one or more zeolites selected from the group consisting of Silicalite 2, ZSM-11, Boralite D, TS-2, SSZ-46, |DEOTA|[Si—B—O]-MEL, and mixtures of two or more thereof, more preferably from the group consisting of Silicalite 2, ZSM-11, TS-2, SSZ-46, and mixtures of two or more thereof, wherein more preferably the zeolitic material comprises ZSM-11 and/or SSZ-46, preferably ZSM-11, wherein more preferably the zeolitic material is ZSM-11 and/or SSZ-46, preferably ZSM-11.

No particular restriction applies as regards the YO₂:X₂O₃ molar ratio of the framework of the zeolitic material. It is preferred that the framework of the zeolitic material displays a YO₂:X₂O₃ molar ratio in the range of from 5 to 300, more preferably from 10 to 200, more preferably from 15 to 150, more preferably from 20 to 120, more preferably from 25 to 100, more preferably from 30 to 80, more preferably from 35 to 70, more preferably from 40 to 60, and more preferably from 45 to 55.

Alternatively, it is preferred that the framework of the zeolitic material displays a YO₂:X₂O₃ molar ratio in the range of from 50 to 1,000, more preferably from 100 to 500, more preferably from 150 to 350, more preferably from 180 to 280, more preferably from 200 to 250, and more preferably from 210 to 230.

It is preferred that Y is selected from the group consisting of Si, Sn, Ti, Zr, Ge, and mixtures of two or more thereof, Y more preferably being Si.

Alternatively, it is preferred that Y comprises Si and Ti, wherein more preferably Y is Si and Ti.

In the case where Y comprises Si and Ti or where Y is Si and Ti, it is preferred that the catalyst provided in (i) and contacted with the mixture in the liquid phase in (iii) displays a Lewis acidity in the range of from 1 to 70, preferably from 2 to 50, more preferably from 3 to 40, more preferably from 4 to 30, more preferably from 5 to 25, more preferably from 6 to 20, more preferably from 7 to 15, more preferably from 8 to 12, and more preferably from 9 to 10.

It is preferred that X is selected from the group consisting of Al, B, In, Ga, and mixtures of two or more thereof, X more preferably being Al and/or B, and more preferably being Al.

It is preferred that the zeolitic material provided in (i) contains one or more transition metal elements, wherein the catalyst provided in (i) is obtained and/or obtainable by a process comprising loading one or more salts of the one or more transition metal elements into the pores of the porous structure of the zeolitic material and optionally on the surface of the zeolitic material.

In the case where the zeolitic material provided in (i) contains one or more transition metal elements, wherein the catalyst provided in (i) is obtained and/or obtainable by a process comprising loading one or more salts of the one or more transition metal elements into the pores of the porous structure of the zeolitic material and optionally on the surface of the zeolitic material, it is preferred that the one or more transition metal elements are selected from the group consisting of Sc, Y, La, Hf, Ce, Dy, Er, Eu, Gd, Ho, La, Lu, Nd, Pr, Pm, Sm, Sc, Tb, Tm, Yb, Fe, Co, Ni, and combinations of two or more thereof,

more preferably from the group consisting of Sc, Y, La, Hf, Ce, Fe, Co, Ni, and combinations of two or more thereof,
more preferably from the group consisting of Sc, La, Hf, Ce, Fe, and combinations of two or more thereof, and
more preferably from the group consisting of Sc, La, Hf, Ce, and combinations of two or more thereof.

It is preferred that the zeolitic material contains 0.1 to 15 weight-% of the one or more transition metal elements, calculated as the element and based on 100 weight-% of YO₂ contained in the framework structure of the zeolitic material, more preferably from 0.5 to 12 weight-%, more preferably from 1 to 10 weight-%, more preferably from 2 to 9 weight-%, more preferably from 3 to 8 weight-%, more preferably from 3.5 to 7 weight-%, more preferably from 4 to 6 weight-%, and more preferably from 4.5 to 5.5 weight-%.

It is preferred that the zeolitic material contains substantially no Na, more preferably substantially no Na or K, more preferably substantially no alkali metal, and more preferably substantially no alkali metal or alkaline earth metal.

No particular restriction applies as regards the Lewis acidity of the catalyst provided in (i) and contacted with the mixture in the liquid phase in (iii). It is preferred that the catalyst provided in (i) and contacted with the mixture in the liquid phase in (iii) displays a Lewis acidity in the range of from 50 to 120, more preferably from 60 to 110, more preferably from 65 to 105, more preferably from 70 to 100, more preferably from 72 to 98, more preferably from 74 to 95, more preferably from 76 to 92, more preferably from 78 to 90, more preferably from 80 to 88, more preferably from 82 to 86, and more preferably from 83 to 84. The Lewis acidity is preferably determined as disclosed herein, in particular as disclosed in the Experimental section.

No particular restriction applies as regards the Brønsted acidity of the catalyst provided in (i) and contacted with the mixture in the liquid phase in (iii). It is preferred that the catalyst provided in (i) and contacted with the mixture in the liquid phase in (iii) displays a Brønsted acidity in the range of from 2 to 70, more preferably from 4 to 65, more preferably from 6 to 60, more preferably from 8 to 55, more preferably from 10 to 50, more preferably from 15 to 45, more preferably from 20 to 40, and more preferably from 25 to 35. The Brønsted acidity is preferably determined as disclosed herein, in particular as disclosed in the Experimental section.

It is preferred that the ratio L:B of the Lewis acidity (L) to the Brønsted acidity (B) of the catalyst provided in (i) and contacted with the mixture in the liquid phase in (iii) is in the range of from 0.5 to 15, more preferably from 0.5 to 12, more preferably from 1 to 9, more preferably from 1 to 7, more preferably from 1.5 to 6, more preferably from 2 to 5, more preferably from 2.5 to 4.5, and more preferably from 3 to 4.

No particular restriction applies as regards the total amount of acid sites of the catalyst provided in (i) and contacted with the mixture in the liquid phase in (iii). It is preferred that the catalyst provided in (i) and contacted with the mixture in the liquid phase in (iii) displays a total amount of acid sites as determined by NH₃-TPD in the range of from 0.1 to 2 mmol/g, more preferably from 0.3 to 1.5 mmol/g, more preferably from 0.4 to 1.2 mmol/g, more preferably from 0.5 to 1 mmol/g, more preferably from 0.55 to 0.9 mmol/g, more preferably from 0.58 to 0.8 mmol/g, more preferably from 0.6 to 0.75 mmol/g, more preferably from 0.63 to 0.72 mmol/g, more preferably from 0.65 to 0.7 mmol/g, and more preferably from 0.67 to 0.68 mmol/g. The total amount of acid sites of the catalyst provided in (i) and contacted with the mixture in the liquid phase in (iii) is preferably determined as disclosed herein, in particular as disclosed in the Experimental section.

No particular restriction applies as regards the amount of weak acid sites of the catalyst provided in (i) and contacted with the mixture in the liquid phase in (iii). It is preferred that the catalyst provided in (i) and contacted with the mixture in the liquid phase in (iii) displays an amount of weak acid sites as determined by NH₃-TPD in the range of from 0.1 to 0.9 mmol/g, more preferably from 0.2 to 0.7 mmol/g, more preferably from 0.3 to 0.6 mmol/g, more preferably from 0.35 to 0.5 mmol/g, more preferably from 0.38 to 0.46 mmol/g, and more preferably from 0.4 to 0.44 mmol/g. The amount of weak acid sites of the catalyst provided in (i) and contacted with the mixture in the liquid phase in (iii) is preferably determined as disclosed herein, in particular as disclosed in the Experimental section.

No particular restriction applies as regards the amount of medium acid sites of the catalyst provided in (i) and contacted with the mixture in the liquid phase in (iii). It is preferred that the catalyst provided in (i) and contacted with the mixture in the liquid phase in (iii) displays an amount of medium acid sites as determined by NH₃-TPD in the range of from 0.01 to 0.5 mmol/g, more preferably from 0.05 to 0.45 mmol/g, more preferably from 0.1 to 0.4 mmol/g, more preferably from 0.15 to 0.35 mmol/g, more preferably from 0.2 to 0.32 mmol/g, more preferably from 0.22 to 0.29 mmol/g, and more preferably from 0.24 to 0.27 mmol/g. The amount of medium acid sites of the catalyst provided in (i) and contacted with the mixture in the liquid phase in (iii) is preferably determined as disclosed herein, in particular as disclosed in the Experimental section.

No particular restriction applies as regards the amount of strong acid sites of the catalyst provided in (i) and contacted with the mixture in the liquid phase in (iii). It is preferred that the catalyst provided in (i) and contacted with the mixture in the liquid phase in (iii) displays an amount of strong acid sites as determined by NH₃-TPD of 0.05 mmol/g or less, more preferably of 0.01 mmol/g or less, more preferably of 0.005 mmol/g or less, more preferably of 0.001 mmol/g or less, more preferably of 0.0005 mmol/g or less, more preferably of 0.0001 mmol/g or less, more preferably of 0.00005 mmol/g or less, and more preferably of 0.00001 mmol/g or less. The amount of strong acid sites of the catalyst provided in (i) and contacted with the mixture in the liquid phase in (iii) is preferably determined as disclosed herein, in particular as disclosed in the Experimental section.

It is preferred that the molar ratio of weak acid sites to medium acid sites as respectively determined by NH₃-TPD of the catalyst provided in (i) and contacted with the mixture in the liquid phase in (iii) is in the range of from 0.1 to 5, more preferably from 0.5 to 3, more preferably from 0.8 to 2.8, more preferably from 1 to 2.5, more preferably from 1.3 to 2, more preferably from 1.5 to 1.8, and more preferably from 1.6 to 1.7.

It is preferred that the BET surface area of the catalyst provided in (i) and contacted with the mixture in the liquid phase in (iii) as determined according to ISO 9277:2010 is in the range of from 100 to 600 m²/g, more preferably from 150 to 500 m²/g, more preferably from 200 to 450 m²/g, more preferably from 225 to 425 m²/g, more preferably from 250 to 400 m²/g, more preferably from 275 to 375 m²/g, more preferably from 300 to 350 m²/g, and more preferably from 315 to 335 m²/g.

It is preferred that the loading of the one or more salts of the one or more transition metal elements into the pores of the porous structure of the zeolitic material and optionally on the surface of the zeolitic material comprises

(a) impregnating the porous structure of the zeolitic material with a solution of the one or more salts of the one or more transition metal elements;
(b) optionally drying the impregnated zeolitic material obtained in (b);
(c) calcining the zeolitic material obtained in (a) or (b).

In the case where the loading of the one or more salts of the one or more transition metal elements into the pores of the porous structure of the zeolitic material and optionally on the surface of the zeolitic material comprises (a), (b), and (c), it is preferred that the solution is an aqueous solution, wherein more preferably the solution consists of the one or more salts of the one or more transition metal elements dissolved in distilled water.

Further in the case where the loading of the one or more salts of the one or more transition metal elements into the pores of the porous structure of the zeolitic material and optionally on the surface of the zeolitic material comprises (a), (b), and (c), it is preferred that the volume of the solution employed in (a) is equal to 500% or less of the total pore volume of the zeolitic material prior to impregnation with the solution, wherein preferably the volume of the solution employed in (a) is equal to 50 to 350% of the total pore volume of the zeolitic material prior to impregnation with the solution, more preferably to 100 to 300%, more preferably to 150 to 270%, more preferably to 180 to 250%, more preferably to 200 to 230%, and more preferably to 210 to 220%, wherein the total pore volume is determined by nitrogen adsorption from the BJH method, preferably according to DIN 66134.

Further in the case where the loading of the one or more salts of the one or more transition metal elements into the pores of the porous structure of the zeolitic material and optionally on the surface of the zeolitic material comprises (a), (b), and (c), it is preferred that (a) is conducted at a temperature in the range of from 5 to 40° C., more preferably from 10 to 35° C., more preferably from 15 to 30° C., and more preferably from 20 to 25° C.

Further in the case where the loading of the one or more salts of the one or more transition metal elements into the pores of the porous structure of the zeolitic material and optionally on the surface of the zeolitic material comprises (a), (b), and (c), it is preferred that the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.

It is preferred that the loading of the one or more salts of the one or more transition metal elements into the pores of the porous structure of the zeolitic material and optionally on the surface of the zeolitic material comprises

(a′) preparing a mixture of the one or more salts of the one or more transition metal elements and the zeolitic material;
(b′) optionally milling the mixture obtained in (a′);
(c′) calcining the zeolitic material obtained in (a′) or (b′).

In the case where that the loading of the one or more salts of the one or more transition metal elements into the pores of the porous structure of the zeolitic material and optionally on the surface of the zeolitic material comprises (a′), (b′), and (c′), it is preferred that the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.

In the case where the loading of the one or more salts of the one or more transition metal elements into the pores of the porous structure of the zeolitic material and optionally on the surface of the zeolitic material comprises (c) or (c′), it is preferred that calcining in (c) or (c′) is conducted at a temperature in the range of from 300 to 900° C., more preferably of from 350 to 700° C., more preferably of from 400 to 600° C., and more preferably of from 450 to 550° C.

Further in the case where the loading of the one or more salts of the one or more transition metal elements into the pores of the porous structure of the zeolitic material and optionally on the surface of the zeolitic material comprises (c) or (c′), it is preferred that calcining in (c) or (c′) is conducted in air.

In the case where the loading of the one or more salts of the one or more transition metal elements into the pores of the porous structure of the zeolitic material and optionally on the surface of the zeolitic material comprises (a) or (a′), it is preferred that prior to the loading of the one or more salts of the one or more transition metal elements into the pores of the porous structure of the zeolitic material and optionally on the surface of the zeolitic material in (a) or (a′), the zeolitic material is in the H-form and contains protons as extra-framework ions, wherein 0.1 weight-% or less of the extra-framework ions are metal cations, calculated as the element and based on 100 weight-% of YO₂ contained in the zeolitic material, more preferably 0.05 weight-% or less, more preferably 0.001 weight-% or less, more preferably 0.0005 weight-% or less, and more preferably 0.0001 weight-% or less.

It is preferred that the catalyst provided in (i) and contacted with the mixture in the liquid phase in (iii) is obtained and/or obtainable by a process which does not comprise a step of ion exchanging the one or more transition metal elements into the zeolitic material.

It is preferred that contacting in (iii) is effected at a temperature in the range of from 40 to 180° C., more preferably from 50 to 150° C., more preferably from 55 to 130° C., more preferably from 60 to 120° C., more preferably from 65 to 115° C., more preferably from 70 to 110° C., more preferably from 75 to 105° C., more preferably from 80 to 100° C., and more preferably from 85 to 95° C.

It is preferred that contacting in (iii) is effected at a pressure in the range of from 50 to 250 bar, more preferably of from 80 to 200 bar, more preferably of from 100 to 180 bar, more preferably of from 110 to 170 bar, more preferably of from 120 to 150 bar, more preferably of from 125 to 145 bar, and more preferably of from 130 to 140 bar.

It is preferred that the ammonia:propylene oxide molar ratio in the mixture in the liquid phase provided in (ii) and contacted with the catalyst in (iii) is in the range of from 1 to 30, more preferably from 3 to 25, more preferably from 5 to 20, more preferably from 6 to 15, more preferably from 7 to 13, more preferably from 7.5 to 11, more preferably from 8 to 10, and more preferably from 8.5 to 9.5.

It is preferred that the weight ratio H₂O:NH₃ of water to ammonia in the mixture in the liquid phase provided in (ii) and contacted with the catalyst in (iii) is in the range of from 0 to 30, more preferably of from 0 to 20, more preferably of from 0 to 15, more preferably of from 0 to 10, more preferably of from 0 to 7, more preferably of from 0 to 5, more preferably of from 0 to 3, more preferably of from 0 to 2, and more preferably of from 0 to 1.

It is preferred that the mixture in the liquid phase provided in (ii) and contacted with the catalyst in (iii) consists of 50 weight-% or more of ammonia and propylene oxide, more preferably 60 weight-% or more, more preferably 70 weight-% or more, more preferably 80 weight-% or more, more preferably 90 weight-% or more, more preferably 95 weight-% or more, more preferably 99 weight-% or more, and more preferably 99.9 weight-% or more.

The unit bar or bar(abs) refers to an absolute pressure of 10⁵ Pa.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A process for the conversion of propylene oxide to 1-amino-2-propanol and/or di(2-hydroxypropyl)amine comprising
   (i) providing a catalyst comprising a zeolitic material comprising YO₂ and optionally comprising X₂O₃ in its framework structure, wherein Y is a tetravalent element and X is a trivalent element, wherein the zeolitic material has a framework-type structure selected from the group consisting of MFI and/or MEL, including MEL/MFI intergrowths;
   (ii) providing a mixture in the liquid phase comprising propylene oxide and ammonia;
   (iii) contacting the catalyst provided in (i) with the mixture in the liquid phase provided in (ii) for converting propylene oxide to 1-amino-2-propanol and/or di(2-hydroxypropyl)amine.
2. The process of embodiment 1, wherein the zeolitic material has an MFI or an MEL/MFI intergrowth framework-type structure, wherein more preferably the zeolitic material has an MFI framework-type structure.
3. The process of embodiment 1 or 2, wherein the catalyst provided in (i) comprises a zeolitic material having an MFI framework-type structure, wherein the zeolitic material preferably comprises one or more zeolites selected from the group consisting of Silicalite, ZSM-5, [Fe—Si—O]-MFI, Monoclinic H-ZSM-5, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, organic-free ZSM-5, and mixtures of two or more thereof, more preferably from the group consisting of ZSM-5, AMS-1B, AZ-1, FZ-1, LZ-105, NU-4, NU-5, TS-1, TSZ, TSZ-III, TZ-01, USC-4, USI-108, ZBH, ZKQ-1B, ZMQT-B, and mixtures of two or more thereof, wherein more preferably the zeolitic material comprises TS-1 and/or ZSM-5, wherein more preferably the zeolitic material is TS-1 and/or ZSM-5.
4. The process of any one of embodiments 1 to 3, wherein the catalyst provided in (i) comprises a zeolitic material having an MEL/MFI intergrowth framework-type structure, wherein the zeolitic material preferably comprises Bor-D and/or ZBM-10, preferably ZBM-10, wherein more preferably the zeolitic material is ZBM-10.
5. The process of any one of embodiments 1 to 4, wherein the catalyst provided in (i) comprises a zeolitic material having an MEL framework-type structure, wherein the zeolitic material preferably comprises one or more zeolites selected from the group consisting of Silicalite 2, ZSM-11, Boralite D, TS-2, SSZ-46, |DEOTA|[Si—B—O]-MEL, and mixtures of two or more thereof, more preferably from the group consisting of Silicalite 2, ZSM-11, TS-2, SSZ-46, and mixtures of two or more thereof, wherein more preferably the zeolitic material comprises ZSM-11 and/or SSZ-46, preferably ZSM-11, wherein more preferably the zeolitic material is ZSM-11 and/or SSZ-46, preferably ZSM-11.
6. The process of any one of embodiments 1 to 5, wherein the framework of the zeolitic material displays a YO₂:X₂O₃ molar ratio in the range of from 5 to 300, preferably from 10 to 200, more preferably from 15 to 150, more preferably from 20 to 120, more preferably from 25 to 100, more preferably from 30 to 80, more preferably from 35 to 70, more preferably from 40 to 60, and more preferably from 45 to 55.
7. The process of any one of embodiments 1 to 5, wherein the framework of the zeolitic material displays a YO₂:X₂O₃ molar ratio in the range of from 50 to 1,000, more preferably from 100 to 500, more preferably from 150 to 350, more preferably from 180 to 280, more preferably from 200 to 250, and more preferably from 210 to 230.
8. The process of any one of embodiments 1 to 7, wherein Y is selected from the group consisting of Si, Sn, Ti, Zr, Ge, and mixtures of two or more thereof, Y preferably being Si.
9. The process of any one of embodiments 1 to 7, wherein Y comprises Si and Ti, wherein more preferably Y is Si and Ti.
10. The process of embodiment 9, wherein the catalyst provided in (i) and contacted with the mixture in the liquid phase in (iii) displays a Lewis acidity in the range of from 1 to 70, preferably from 2 to 50, more preferably from 3 to 40, more preferably from 4 to 30, more preferably from 5 to 25, more preferably from 6 to 20, more preferably from 7 to 15, more preferably from 8 to 12, and more preferably from 9 to 10.
11. The process of any one of embodiments 1 to 10, wherein X is selected from the group consisting of Al, B, In, Ga, and mixtures of two or more thereof, X preferably being Al and/or B, and more preferably being Al.
12. The process of any one of embodiments 1 to 11, wherein the zeolitic material provided in (i) contains one or more transition metal elements, wherein the catalyst provided in (i) is obtained and/or obtainable by a process comprising loading one or more salts of the one or more transition metal elements into the pores of the porous structure of the zeolitic material and optionally on the surface of the zeolitic material.
13. The process of embodiment 12, wherein the one or more transition metal elements are selected from the group consisting of Sc, Y, La, Hf, Ce, Dy, Er, Eu, Gd, Ho, La, Lu, Nd, Pr, Pm, Sm, Sc, Tb, Tm, Yb, Fe, Co, Ni, and combinations of two or more thereof,
   preferably from the group consisting of Sc, Y, La, Hf, Ce, Fe, Co, Ni, and combinations of two or more thereof,
   more preferably from the group consisting of Sc, La, Hf, Ce, Fe, and combinations of two or more thereof, and
   more preferably from the group consisting of Sc, La, Hf, Ce, and combinations of two or more thereof.
14. The process of any one of embodiments 1 to 13, wherein the zeolitic material contains 0.1 to 15 weight-% of the one or more transition metal elements, calculated as the element and based on 100 weight-% of YO₂ contained in the framework structure of the zeolitic material, preferably from 0.5 to 12 weight-%, more preferably from 1 to 10 weight-%, more preferably from 2 to 9 weight-%, more preferably from 3 to 8 weight-%, more preferably from 3.5 to 7 weight-%, more preferably from 4 to 6 weight-%, and more preferably from 4.5 to 5.5 weight-%.
15. The process of any one of embodiments 1 to 14, wherein the zeolitic material contains substantially no Na, preferably substantially no Na or K, more preferably substantially no alkali metal, and more preferably substantially no alkali metal or alkaline earth metal.
16. The process of any one of embodiments 1 to 15, wherein the catalyst provided in (i) and contacted with the mixture in the liquid phase in (iii) displays a Lewis acidity in the range of from 50 to 120, preferably from 60 to 110, more preferably from 65 to 105, more preferably from 70 to 100, more preferably from 72 to 98, more preferably from 74 to 95, more preferably from 76 to 92, more preferably from 78 to 90, more preferably from 80 to 88, more preferably from 82 to 86, and more preferably from 83 to 84.
17. The process of any one of embodiments 1 to 16, wherein the catalyst provided in (i) and contacted with the mixture in the liquid phase in (iii) displays a Brønsted acidity in the range of from 2 to 70, preferably from 4 to 65, more preferably from 6 to 60, more preferably from 8 to 55, more preferably from 10 to 50, more preferably from 15 to 45, more preferably from 20 to 40, and more preferably from 25 to 35.
18. The process of any one of embodiments 1 to 17, wherein the ratio L:B of the Lewis acidity (L) to the Brønsted acidity (B) of the catalyst provided in (i) and contacted with the mixture in the liquid phase in (iii) is in the range of from 0.5 to 15, preferably from 0.5 to 12, more preferably from 1 to 9, more preferably from 1 to 7, more preferably from 1.5 to 6, more preferably from 2 to 5, more preferably from 2.5 to 4.5, and more preferably from 3 to 4.
19. The process of any one of embodiments 1 to 18, wherein the catalyst provided in (i) and contacted with the mixture in the liquid phase in (iii) displays a total amount of acid sites as determined by NH₃-TPD in the range of from 0.1 to 2 mmol/g, preferably from 0.3 to 1.5 mmol/g, more preferably from 0.4 to 1.2 mmol/g, more preferably from 0.5 to 1 mmol/g, more preferably from 0.55 to 0.9 mmol/g, more preferably from 0.58 to 0.8 mmol/g, more preferably from 0.6 to 0.75 mmol/g, more preferably from 0.63 to 0.72 mmol/g, more preferably from 0.65 to 0.7 mmol/g, and more preferably from 0.67 to 0.68 mmol/g.
20. The process of any one of embodiments 1 to 19, wherein the catalyst provided in (i) and contacted with the mixture in the liquid phase in (iii) displays an amount of weak acid sites as determined by NH₃-TPD in the range of from 0.1 to 0.9 mmol/g, preferably from 0.2 to 0.7 mmol/g, more preferably from 0.3 to 0.6 mmol/g, more preferably from 0.35 to 0.5 mmol/g, more preferably from 0.38 to 0.46 mmol/g, and more preferably from 0.4 to 0.44 mmol/g.
21. The process of any one of embodiments 1 to 20, wherein the catalyst provided in (i) and contacted with the mixture in the liquid phase in (iii) displays an amount of medium acid sites as determined by NH₃-TPD in the range of from 0.01 to 0.5 mmol/g, preferably from 0.05 to 0.45 mmol/g, more preferably from 0.1 to 0.4 mmol/g, more preferably from 0.15 to 0.35 mmol/g, more preferably from 0.2 to 0.32 mmol/g, more preferably from 0.22 to 0.29 mmol/g, and more preferably from 0.24 to 0.27 mmol/g.
22. The process of any one of embodiments 1 to 21, wherein the catalyst provided in (i) and contacted with the mixture in the liquid phase in (iii) displays an amount of strong acid sites as determined by NH₃-TPD of 0.05 mmol/g or less, more preferably of 0.01 mmol/g or less, more preferably of 0.005 mmol/g or less, more preferably of 0.001 mmol/g or less, more preferably of 0.0005 mmol/g or less, more preferably of 0.0001 mmol/g or less, more preferably of 0.00005 mmol/g or less, and more preferably of 0.00001 mmol/g or less.
23. The process of any one of embodiments 1 to 22, wherein the molar ratio of weak acid sites to medium acid sites as respectively determined by NH₃-TPD of the catalyst provided in (i) and contacted with the mixture in the liquid phase in (iii) is in the range of from 0.1 to 5, preferably from 0.5 to 3, more preferably from 0.8 to 2.8, more preferably from 1 to 2.5, more preferably from 1.3 to 2, more preferably from 1.5 to 1.8, and more preferably from 1.6 to 1.7.
24. The process of any one of embodiments 1 to 23, wherein the BET surface area of the catalyst provided in (i) and contacted with the mixture in the liquid phase in (iii) as determined according to ISO 9277:2010 is in the range of from 100 to 600 m²/g, preferably from 150 to 500 m²/g, more preferably from 200 to 450 m²/g, more preferably from 225 to 425 m²/g, more preferably from 250 to 400 m²/g, more preferably from 275 to 375 m²/g, more preferably from 300 to 350 m²/g, and more preferably from 315 to 335 m²/g.
25. The process of any one of embodiments 1 to 24, wherein the loading of the one or more salts of the one or more transition metal elements into the pores of the porous structure of the zeolitic material and optionally on the surface of the zeolitic material comprises
   (a) impregnating the porous structure of the zeolitic material with a solution of the one or more salts of the one or more transition metal elements;
   (b) optionally drying the impregnated zeolitic material obtained in (b);
   (c) calcining the zeolitic material obtained in (a) or (b).
26. The process of embodiment 25, wherein the solution is an aqueous solution, wherein preferably the solution consists of the one or more salts of the one or more transition metal elements dissolved in distilled water.
27. The process of embodiment 25 or 26, wherein the volume of the solution employed in (a) is equal to 500% or less of the total pore volume of the zeolitic material prior to impregnation with the solution, wherein preferably the volume of the solution employed in (a) is equal to 50 to 350% of the total pore volume of the zeolitic material prior to impregnation with the solution, more preferably to 100 to 300%, more preferably to 150 to 270%, more preferably to 180 to 250%, more preferably to 200 to 230%, and more preferably to 210 to 220%, wherein the total pore volume is determined by nitrogen adsorption from the BJH method, preferably according to DIN 66134.
28. The process of any one of embodiments 25 to 27, wherein (a) is conducted at a temperature in the range of from 5 to 40° C., preferably from 10 to 35° C., more preferably from 15 to 30° C., and more preferably from 20 to 25° C.
29. The process of any one of embodiments 25 to 28, wherein the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.
30. The process of any one of embodiments 1 to 24, wherein the loading of the one or more salts of the one or more transition metal elements into the pores of the porous structure of the zeolitic material and optionally on the surface of the zeolitic material comprises
   (a′) preparing a mixture of the one or more salts of the one or more transition metal elements and the zeolitic material;
   (b′) optionally milling the mixture obtained in (a′);
   (c′) calcining the zeolitic material obtained in (a′) or (b′).
31. The process of embodiment 30, wherein the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.
32. The process of any one of embodiments 25 to 31, wherein calcining in (c) or (c′) is conducted at a temperature in the range of from 300 to 900° C., preferably of from 350 to 700° C., more preferably of from 400 to 600° C., and more preferably of from 450 to 550° C.
33. The process of any one of embodiments 25 to 32, wherein calcining in (c) or (c′) is conducted in air.
34. The process of any one of embodiments 25 to 33, wherein prior to the loading of the one or more salts of the one or more transition metal elements into the pores of the porous structure of the zeolitic material and optionally on the surface of the zeolitic material in (a) or (a′), the zeolitic material is in the H-form and contains protons as extra-framework ions, wherein 0.1 weight-% or less of the extra-framework ions are metal cations, calculated as the element and based on 100 weight-% of YO₂ contained in the zeolitic material, preferably 0.05 weight-% or less, more preferably 0.001 weight-% or less, more preferably 0.0005 weight-% or less, and more preferably 0.0001 weight-% or less.
35. The process of any one of embodiments 1 to 34, wherein the catalyst provided in (i) and contacted with the mixture in the liquid phase in (iii) is obtained and/or obtainable by a process which does not comprise a step of ion exchanging the one or more transition metal elements into the zeolitic material.
36. The process of any one of embodiments 1 to 35, wherein the contacting in (iii) is effected at a temperature in the range of from 40 to 180° C., preferably from 50 to 150° C., more preferably from 55 to 130° C., more preferably from 60 to 120° C., more preferably from 65 to 115° C., more preferably from 70 to 110° C., more preferably from 75 to 105° C., more preferably from 80 to 100° C., and more preferably from 85 to 95° C.
37. The process of any one of embodiments 1 to 36, wherein the contacting in (iii) is effected at a pressure in the range of from 50 to 250 bar, preferably of from 80 to 200 bar, more preferably of from 100 to 180 bar, more preferably of from 110 to 170 bar, more preferably of from 120 to 150 bar, more preferably of from 125 to 145 bar, and more preferably of from 130 to 140 bar.
38. The process of any one of embodiments 1 to 37, wherein the ammonia:propylene oxide molar ratio in the mixture in the liquid phase provided in (ii) and contacted with the catalyst in (iii) is in the range of from 1 to 30, preferably from 3 to 25, more preferably from 5 to 20, more preferably from 6 to 15, more preferably from 7 to 13, more preferably from 7.5 to 11, more preferably from 8 to 10, and more preferably from 8.5 to 9.5.
39. The process of any one of embodiments 1 to 38, wherein the weight ratio H₂O:NH₃ of water to ammonia in the mixture in the liquid phase provided in (ii) and contacted with the catalyst in (iii) is in the range of from 0 to 30, preferably of from 0 to 20, more preferably of from 0 to 15, more preferably of from 0 to 10, more preferably of from 0 to 7, more preferably of from 0 to 5, more preferably of from 0 to 3, more preferably of from 0 to 2, and more preferably of from 0 to 1.
40. The process of any one of embodiments 1 to 39, wherein the mixture in the liquid phase provided in (ii) and contacted with the catalyst in (iii) consists of 50 weight-% or more of ammonia and propylene oxide, preferably 60 weight-% or more, more preferably 70 weight-% or more, more preferably 80 weight-% or more, more preferably 90 weight-% or more, more preferably 95 weight-% or more, more preferably 99 weight-% or more, and more preferably 99.9 weight-% or more.

### EXPERIMENTAL SECTION

The present invention is further illustrated by the following Reference Examples, Examples, and Comparative Examples.

### Determination of the Bronsted and Lewis Acidities

In the examples, the Bronsted and Lewis acidities were determined using pyridine as the probe gas. The measurements were conducted using an IR-spectrometer Nicolet 6700 employing a HV-FTIR-cell. The samples were pressed to a pellet for placing in the HV-FTIR-cell for measurement. After being placed in the HV-FTIR-cell, the samples were then heated in air to 350° C. and held at that temperature for 1 h for removing water and any volatile substances from the sample. The apparatus was then placed under high-vacuum (10⁻⁵ mbar), and the cell let cool to 80° C., at which it was held for the entire duration of the measurement for avoiding the condensation of pyridine in the cell.

Pyridine was then dosed into the cell in successive steps (0.01, 0.1, 1, and 3 mbar) to ensure the controlled and complete exposition of the sample.

The irradiation spectrum of the activated sample at 80° C. and 10⁻⁵ mbar was used as the background for the absorbtion spectra for compensating the influence of matrix bands.

For the analysis, the spectrum at a pressure of 1 mbar was used, since the sample was in a stable equilibrium. For the quantification, the extinction spectrum was used, since this allowed for the cancellation of the matrix effects.

The integral extinction unit was determined as follows: the characteristic signals for the pyridine absorption were integrated and the area of the pellet was scaled with the thickness of the pellet.

Overview table: Assignment of the IR-bands of pyridine

| acid sites | pyridine species | bands (cm⁻¹) |
|---|---|---|
| L | Py | 1440-1455 |
| | | 1575 |
| | | 1620 |
| B | PyH⁺ | 1540-1550 |
| | | 1635-1640 |
| B + L | Py + PyH⁺ | 1490 |
| physical adsorbate | adsorbated Py | 1440 (overlay L) |
| | | 1580-1595 |

Py = pyridine;
PyH⁺ = pyridinium ion;
B = Bronsted center;
L = Lewis center

In the examples, the determination of the Lewis acid sites were determined using the band at 1450 cm⁻¹ and of the Bronsted acid sites using the band at 1545 cm⁻¹.

### Temperature Programmed Desorption of Ammonia (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).
2. Saturation with NH₃: Commencement of recording; one measurement per second.

Change the gas flow to a mixture of 10% NH₃ in He (75 cm³/min; 100° C. and 1 atm) at 100° C.; hold for 30 minutes.
3. Removal of the excess: Commencement of recording; one measurement per second. Change the gas flow to a He flow of 75 cm³/min (100° C. and 1 atm) at 100° C.; hold for 60 min.
4. NH₃-TPD: Commencement of recording; one measurement per second. Heat up under a He flow (flow rate: 30 cm³/min) to 600° C. at a heating rate of 10 K/min; hold for 30 minutes.
5. End of measurement.

Desorbed ammonia was measured by means of the online mass spectrometer, which demonstrates that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Reference Example 1: Preparation of H-ZSM-5 (MFI-Type Framework Structure)

In a 2 m³ reactor 79.61 kg of de-ionised water is first introduced. To the water, 411.15 kg of an aqueous tetrapropylammonium hydroxide solution (TPAOH; 40 wt. %) was added under stirring (70 rpm). The suspension is let for stirring for another 10 min. 8.2 kg solid NaOH is added slowly in 2.5 kg portions under stirring and after each portion the system is allowed to mix for 5 minutes. Next, 29.25 kg aluminium triisopropoxide is added to the suspension and the system is stirred for another 1 h. At the end, 538.19 kg colloidal silica (Ludox AS-40) is added followed by additional 10 kg of de-ionized water. The synthesis mixture is stirred another 1 h at room temperature before the reactor is flushed with nitrogen gas and the pressure reduced to 900 mbar.

Afterwards the reactor is heated to 170° C. in 11 h. The hydrothermal synthesis is run for 72 h at 170° C. under 70 rpm stirring. After crystallization the synthesis mixture is cooled down to 30° C. The suspension is transferred to a larger vessel where the pH of the suspension is adjusted to 7±0.5, by addition of a 10 wt. % aqueous nitric acid solution. The pH adjusted suspension is let for stirring for another 30 min at 70 rpm. The zeolite is separated by filtration and the filter cake is washed with de-ionised water until a conductivity of the wash water <200 μS. The filtercake is then dried at 120° C. for 96 h. The dried material was calcined to 550° C. in air for 6 h for obtaining a calcined ZSM-5 zeolite with a BET surface area of 390 m²/g, and displaying a crystallinity as determined by X-ray diffraction of 94%.

250 kg de-ionized water is added to a 400 L reactor and 25 kg ammonium nitrate is added under stirring (150 rpm). The suspension is heated to 80° C., followed by the addition of 25 kg of the calcined zeolite. The mixture is stirred further for 1 h at 80° C. Afterwards the reaction mixture is cooled down and filtered off using a filterpress and washed with water until a conductivity in the wash water <200 μS. The ion-exchange process is then repeated for obtaining an ammonium-exchanged ZSM-5. The filter cake obtained after the second ammonium ion-exchange process is dried for 10 h at 120° C. and calcined at 500° C. in air for 5 h (heating rate 2° C./min) for obtaining H-ZSM-5.

According to the elemental analysis the resulting product had the following contents determined per 100 g substance of <0.1 g carbon, 1.6 g aluminum, <0.01 g of sodium, and 43 g silicon.

The BET surface area was determined to be 408 m²/g.

### Reference Example 2: Extrusion of H-ZSM-5 (MW1032)

100.0 g of H-ZSM-5 as obtained according to Reference Example 1 was then admixed with 27.77 g of colloidal silica (Ludox AS-40) and 5.0 g Walocel binder (Wolf Walsrode AG PUFAS Werk KG), wherein the resulting mixture was kneaded for 10 min, after which 80.0 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 91.9 g of the calcined extrudate.

### Reference Example 3: Preparation of La-ZSM-5 by Wet Impregnation and Extrusion Thereof

70.0 g of H-ZSM-5 as obtained according to Reference Example 1 were added to a solution of 13.69 g of La(NO₃)₃.6 H₂O dissolved in 70 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 79.5 g of La-ZSM-5.

The BET surface area was determined to be 318 m²/g.

The 79.5 g La-ZSM-5 was then admixed with 22.08 g of colloidal silica (Ludox AS-40) and 3.9 g Walocel binder (Wolf Walsrode AG PUFAS Werk KG), wherein the resulting mixture was kneaded for 10 min, after which 50 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 66.2 g of the calcined extrudate.

### Reference Example 4: Preparation of Ce-ZSM-5 by Wet Impregnation and Extrusion Thereof

70 g of H-ZSM-5 as obtained according to Reference Example 1 were added to a solution of 26.81 g of Ce(NO₃)₃.6 H₂O dissolved in 70 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 80.5 g of Ce-ZSM-5.

The BET surface area was determined to be 336 m²/g.

The Ce-ZSM-5 was then admixed with 22.36 g of colloidal silica (Ludox AS-40) and 4.03 g Walocel binder (Wolf Walsrode AG PUFAS Werk KG), wherein the resulting mixture was kneaded for 10 min, after which 54 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 66.5 g of the calcined extrudate.

### Reference Example 5: Preparation of Sc-ZSM-5 by Wet Impregnation and Extrusion Thereof

175.0 g of H-ZSM-5 as obtained according to Reference Example 1 were added to a solution of 30.0 g of Sc(NO₃)₃.H₂O dissolved in 175.0 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 182.6 g of Sc-ZSM-5.

75.0 g of Sc-ZSM-5 was then admixed with 20.8 g of colloidal silica (Ludox AS-40) and 3.75 g Walocel binder (Wolf Walsrode AG PUFAS Werk KG), wherein the resulting mixture was kneaded for 10 min, after which 54.0 ml of distilled water were added and the resulting mixture was kneaded for an additional 20 min. The kneaded mixture was than extruded to strands with a diameter of 1.5 mm. The extrudate was then heated to 120° C. at a rate of 3° C./min, held at that temperature for 7 hours, and then heated further to 500° C. at a rate of 2° C./min and calcined at that temperature for 2 h for obtaining 61.0 g of the calcined extrudate.

### Reference Example 6: Preparation of Hf-ZSM-5 by Wet Impregnation and Extrusion Thereof

200.0 g of H-ZSM-5 as obtained according to Reference Example 1 were added to a solution of 18.89 g of HfCl₄ dissolved in 200.0 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 208.3 g of Hf-ZSM-5.

75.0 g of Hf-ZSM-5 was then admixed with 20.8 g of colloidal silica (Ludox AS-40) and 3.75 g Walocel binder (Wolf Walsrode AG PUFAS Werk KG), wherein the resulting mixture was kneaded for 10 min, after which 60.0 ml of distilled water were added and the resulting mixture was kneaded for an additional 20 min. The kneaded mixture was than extruded to strands with a diameter of 1.0 mm. The extrudate was then heated to 120° C. at a rate of 3° C./min, held at that temperature for 7 hours, and then heated further to 500° C. at a rate of 2° C./min and calcined at that temperature for 2 h for obtaining 59.0 g of the calcined extrudate.

### Reference Example 7: Preparation of Fe-ZSM-5 by Wet Impregnation and Extrusion Thereof

200.0 g of H-ZSM-5 as obtained according to Reference Example 1 were added to a solution of 76.14 g of Fe(NO₃)₃*9 H₂O dissolved in 200.0 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 208.0 g of Fe-ZSM-5.

75.0 g of Fe-ZSM-5 was then admixed with 20.8 g of colloidal silica (Ludox AS-40) and 3.75 g Walocel binder (Wolf Walsrode AG PUFAS Werk KG), wherein the resulting mixture was kneaded for 10 min, after which 54.0 ml of distilled water were added and the resulting mixture was kneaded for an additional 20 min. The kneaded mixture was than extruded to strands with a diameter of 1.0 mm. The extrudate was then heated to 120° C. at a rate of 3° C./min, held at that temperature for 7 hours, and then heated further to 500° C. at a rate of 2° C./min and calcined at that temperature for 2 h for obtaining 65.6 g of the calcined extrudate.

### Reference Example 8: Preparation of TS-1 and Extrusion Thereof

An extruded TS-1 material was produced in accordance with the experimental procedure disclosed in WO 2011/064191 A1.

### Examples: Amination of Propylene Oxide

The material from each of Reference Examples 2 to 9 was respectively filtered for obtaining a split fraction in the range of from 0.4-0.8 mm, which was then filled into the reactor (tubular reactor with a length of 1350 mm and a diameter of 0.5 mm (reactor volume=3.66 ml/m), wherein the reactor had a wall thickness of 3.17 mm), and the reactor vessel was then flooded with nitrogen prior to testing.

Propylene oxide and ammonia were continually pumped into a pre-mixing unit (2.0 ml volume) and then introduced into the reactor which was heated to a given temperature for reacting the mixture over the catalyst sample. For the analysis of the product mixture, a sample of 0.25 ml was collected and was quenched in a pressure vessel with HOAc (7.0 ml). For analytical assessment, 0.75 ml of the sample were then transferred to a gas chromatography-phial and then tempered for 16 h at 65° C., after which 0.75 ml of Ac₂O were added and the sample incubated at 65° C. for an additional 16 h. The gas chromatographical analysis was conducted on a 60 m RTX1 column (temperature ramp: 80° C. starting temperature and heating at a rate of 8° C./min to 280° C.) with the following retention times: rₜ (MIPOA)=8.98 min; rₜ (DIPOA)=15.81 min; rₜ (TIPOA)=21.18 min.

TABLE 1
Results from the amination of propylene oxide at a NH₃:PO molar ratio
of 9 using the catalysts from Reference Examples 2 to 9 for different
temperature ranges (39-41, 57-60, 84-90, 99, and 109).

| | | Metal | | Temp. | Conv. | Product distribution | | |
|---|---|---|---|---|---|---|---|---|
| Catalyst | framework | (wt.-%) | SAR | [° C.] | [%] | MIPOA | DIPOA | TIPOA |
| RE 2 | MFI | — | 50 | 40 | 2 | 100 | 0 | 0 |
| RE 3 | MFI | La (4.9) | 50 | 41 | 57 | 83 | 17 | 0 |
| RE 4 | MFI | Ce (8.7) | 50 | 40 | 11 | 96 | 4 | 0 |
| RE 6 | MFI | Hf (5.3) | 50 | 39 | 3 | 100 | 0 | 0 |
| RE 5 | MFI | Sc (2.3) | 50 | 41 | 13 | 97 | 3 | 0 |
| RE 4 | MFI | Ce (8.7) | 50 | 57 | 28 | 89 | 11 | 0 |
| RE 5 | MFI | Sc (2.3) | 50 | 59 | 36 | 91 | 9 | 0 |
| RE 7 | MFI | Fe (4.8) | 50 | 60 | 7 | 97 | 3 | 0 |
| RE 8 | MFI | Ti | — | 60 | 61 | 76 | 24 | 0 |
| RE 2 | MFI | — | 50 | 86 | 21 | 88 | 12 | 0 |
| RE 4 | MFI | Ce (8.7) | 50 | 87 | 92 | 76 | 24 | 0 |
| RE 6 | MFI | Hf (5.3) | 50 | 84 | 34 | 86 | 14 | 0 |
| RE 7 | MFI | Fe (4.8) | 50 | 90 | 34 | 86 | 14 | 0 |
| RE 8 | MFI | Ti | — | 90 | 100 | 71 | 29 | 0 |
| RE 5 | MFI | Sc (2.3) | 50 | 99 | >99 | 80 | 20 | 0 |
| RE 6 | MFI | Hf (5.3) | 50 | 109 | 89 | 74 | 26 | 0 |

TABLE 2
Results from the amination of propylene oxide at different NH₃:PO molar ratios
and temperatures using the catalysts from Reference Examples 3 and 5.

| | | Metal | | Temp. | Conv. | Product distribution | | |
|---|---|---|---|---|---|---|---|---|
| Catalyst | framework | (wt.-%) | NH₃:PO | [° C.] | [%] | MIPOA | DIPOA | TIPOA |
| RE 3 | MFI | La (4.9) | 20 | 73 | >99 | 87 | 13 | 0 |
| | | | 20 | 60 | 84 | 88 | 12 | 0 |
| | | | 4 | 61 | >99 | 62 | 38 | 0 |
| | | | 2 | 60 | >99 | 46 | 53 | 2 |
| RE 5 | MFI | Sc (2.3) | 20 | 103 | 94 | 91 | 9 | 0 |
| | | | 4 | 98 | 99 | 59 | 41 | 0 |
| | | | 3 | 97 | >99 | 49 | 50 | 1 |

TABLE 3
Acidity characteristics of the zeolitic materials of the MFI framework type from
Reference Examples 3-8 as determined from NH₃-TPD and HV-FTIR spectroscopy.

| | | Total | Weak | Medium | Strong | | | |
|---|---|---|---|---|---|---|---|---|
| | | Acid | Acid | Acid | Acid | | | |
| | Metal | Sites | Sites | Sites | Sites | Lewis | Bronsted | Lewis:Bronsted |
| Catalyst | (wt.-%) | [mmol/g] | [mmol/g] | [mmol/g] | [mmol/g] | acidity | acidity | acidity ratio |
| RE 3 | La (4.9) | 0.677 | 0.418 | 0.255 | 0.004 | 86.9 | 18.7 | 4.65 |
| RE 4 | Ce (8.7) | 0.553 | 0.399 | 0.151 | 0.003 | 86.7 | 10.8 | 8.03 |
| RE 5* | Sc (2.3) | — | — | — | — | 80.4 | 42.7 | 1.88 |
| RE 6* | Hf (5.3) | — | — | — | — | 74.8 | 45.3 | 1.65 |
| RE 7* | Fe (4.8) | — | — | — | — | 85.5 | 53.9 | 1.59 |
| RE 8* | Ti | 0.030 | 0.019 | 0.005 | 0.006 | 9.24 | — | — |

*Analytics describe catalyst's composition before extrusion.

Thus, as may be taken from the results displayed in Table 1, high conversion rates and high MIPOA selectivities may be obtained when using the zeolitic materials of the reference examples in the inventive process, in particular in cases in which the material is doped with a transition metal element or when the framework of the zeolitic material is isomorphously substituted with a transition metal element as in the case of Reference Example 9. As may be taken from the results in Table 2, the product distribution may accordingly be influenced by varying the NH₃:PO molar ratio of the reaction mixture, higher ratios accordingly favoring the production of MIPOA. When comparing the results obtained for Reference Example 3 at 41° C. and of Reference Example 4 at 56° C. it is apparent that zeolites doped with a lower SAR (silica to alumina ratio) and amount of La gave particularly better results in catalysis. Also, it is observed that Sc proved to work well at higher temperatures. Hf and Ce, on the other hand, were comparable, however slightly below the activity of Sc and La. Comparing the activity of first row transition-metals it was generally found that early transition metal doped zeolites containing Sc or Ti proved to have a better activity than late transition metal doped zeolites (e.g. Fe).

Interestingly, direct comparison between La and Sc doped zeolites reveals some surprises: Whereas La doped zeolites give good conversions and selectivities at lower temperatures, they lack at higher temperatures, resulting in a drop in selectivity. Sc doped zeolite catalysts exponentially increase in their activity when going to higher temperatures (100° C.), giving both full conversion and high product selectivities.

Consequently, it has unexpectedly been found that a highly efficient process for the amination of propylene oxide may be provided according to the present invention, in particular with regard to both the conversion rate and the selectivities towards MIPOA.

### CITED PRIOR ART

U.S. Pat. No. 3,697,598 A
U.S. Pat. No. 5,599,999 A
U.S. Pat. No. 4,438,281 A
EP 0375267 A2
CN 101884934 A

1.-14. (canceled)

15. A process for the conversion of propylene oxide to 1-amino-2-propanol and/or di(2-hydroxypropyl)amine comprising
(i) providing a catalyst comprising a zeolitic material comprising YO2 and optionally comprising X₂O₃ in its framework structure, wherein Y is a tetravalent element and X is a trivalent element, wherein the zeolitic material has a framework-type structure selected from the group consisting of MFI and/or MEL, including MEL/MFI intergrowths;
(ii) providing a mixture in the liquid phase comprising propylene oxide and ammonia;
(iii) contacting the catalyst provided in (i) with the mixture in the liquid phase provided in (ii) for converting propylene oxide to 1-amino-2-propanol and/or di(2-hydroxypropyl)amine, wherein the catalyst provided in (i) and contacted with the mixture in the liquid phase in (iii) displays a Lewis acidity in the range of from 1 to 70 and wherein Y comprises Si and Ti, or wherein the catalyst provided in (i) and contacted with the mixture in the liquid phase in (iii) displays a Lewis acidity in the range of from 50 to 120.

16. The process of claim 15, wherein the zeolitic material has an MFI or an MEL/MFI inter-growth framework-type structure.

17. The process of claim 15, wherein the framework of the zeolitic material displays a YO2:X2O3 molar ratio in the range of from 5 to 300 or in the range of from 50 to 1,000.

18. The process of claim 15, wherein Y is selected from the group consisting of Si, Sn, Ti, Zr, Ge, and mixtures of two or more thereof.

19. The process of claim 15, wherein X is selected from the group consisting of Al, B, In, Ga, and mixtures of two or more thereof.

20. The process of claim 15, wherein the zeolitic material provided in (i) contains one or more transition metal elements, wherein the catalyst provided in (i) is obtained and/or obtainable by a process comprising loading one or more salts of the one or more transition metal elements into the pores of the porous structure of the zeolitic material and optionally on the surface of the zeolitic material.

21. The process of claim 15, wherein the zeolitic material contains 0.1 to 15 weight-% of the one or more transition metal elements, calculated as the element and based on 100 weight-% of YO2 contained in the framework structure of the zeolitic material.

22. The process of claim 15, wherein the catalyst provided in (i) and contacted with the mixture in the liquid phase in (iii) displays a Brønsted acidity in the range of from 2 to 70.

23. The process of claim 15, wherein the ratio L:B of the Lewis acidity (L) to the Brønsted acidity (B) of the catalyst provided in (i) and contacted with the mixture in the liquid phase in (iii) is in the range of from 0.5 to 15.

24. The process of claim 15, wherein the contacting in (iii) is effected at a temperature in the range of from 40 to 180° C.

25. The process of claim 15, wherein the contacting in (iii) is effected at a pressure in the range of from 50 to 250 bar.

26. The process of claim 15, wherein the ammonia:propylene oxide molar ratio in the mixture in the liquid phase provided in (ii) and contacted with the catalyst in (iii) is in the range of from 1 to 30.

27. The process of claim 15, wherein the weight ratio H2O:NH3 of water to ammonia in the mixture in the liquid phase provided in (ii) and contacted with the catalyst in (iii) is in the range of from 0 to 30.

28. The process of claim 15, wherein the mixture in the liquid phase provided in (ii) and contacted with the catalyst in (iii) consists of 50 weight-% or more of ammonia and propylene oxide.
